(19) United States
(12) Patent Application Publication
(10) Pub. No.: US 2020/0393455 A1
(43) Pub. Date: Dec. 17, 2020

(54) METHODS FOR IDENTIFYING AND QUANTITATING HOST CELL PROTEIN

(71) Applicant: Regeneron Pharmaceuticals, Inc., Tarrytown, NY (US)

(72) Inventors: Nisha Palackal, White Plains, NY (US); Kun Lu, Scarsdale, NY (US); Erica Pyles, New City, NY (US)

(21) Appl. No.: 16/880,736

(22) Filed: May 21, 2020

Related U.S. Application Data
(60) Provisional application No. 62/850,999, filed on May 21, 2019.

(51) Int. Cl. G01N 33/543; G01N 27/447

(52) U.S. Cl. CPC G01N 33/54366; G01N 27/44743; G01N 2800/2828; G01N 27/44795; G01N 33/54306; G01N 27/44726

(57) ABSTRACT

Methods for detecting and/or discriminating between variants of a contaminating protein or multiple contaminating proteins in a sample by a physical parameter, in which the method includes: separating protein components of a sample by molecular weight or charge in one or more capillaries using capillary electrophoresis; immobilizing the protein components of the sample within the one or more capillaries; contacting the protein components within the one or more capillaries with one or more primary antibodies that specifically bind to the contaminating protein or multiple contaminating proteins in the sample, thereby detecting and/or discriminating between variants in the sample.

### FIELD OF THE INVENTION

The present invention pertains to biopharmaceuticals, and relates to the use of capillary electrophoresis to detect contaminant polypeptides in biopharmaceutical preparations, including host cell protein contaminants.

### BACKGROUND

Monoclonal antibodies (mAbs) are a significant class of biotherapeutic products, and they have achieved outstanding success in treating many life-threatening and chronic diseases. However, mAbs are purified from highly complex mixtures of biological macromolecules with size and charge variants, various post translational modifications, including different glycosylation patterns, and N and C terminal heterogeneity. Each individual monoclonal antibody preparation may therefore present a unique profile of host cell proteins, a characteristic which needs to be taken into consideration during the evaluation of these products both during development and manufacturing of final product. For recombinant biopharmaceutical proteins to be acceptable for administration to human patients, it is important that residual impurities resulting from the manufacture and purification process are removed from the final biological product, These process components include culture medium proteins, immunoglobulin affinity ligands, viruses, endotoxin, DNA, and host cell proteins. These host cell impurities include process-specific host cell proteins (HCPs), which are process-related impurities/contaminants in the biologics derived from recombinant DNA technology. While HCPs are typically present in the final drug substance in small quantities (in parts-per-million or nanograms per milligram of the intended recombinant protein), it is recognized that HCPs are undesirable and their quantities should be minimized. For example, the U.S. Food and Drug Administration (FDA) requires that biopharmaceuticals intended for in vivo human use should be as free as possible of extraneous impurities, and requires tests for detection and quantitation of potential contaminants/impurities, such as HCPs. In addition, the International Conference on Harmonization (ICH) provides guidelines on test procedures and acceptance criteria for biotechnological; biological products. The guidelines suggest that for HCPs, a sensitive immunoassay capable of detecting a wide range of protein impurities be utilized.

Sensitive analytical methods, such as LC-MS/MS can be used to identify and quantify single HCP species present in excess of protein components. Upon identification of such single HCP species, alternative assays of sufficient sensitivity and specificity and that are capable of being validated for approval by regulatory authorities and that can be used as a platform across multiple recombinant protein products, need to be developed.

Electrophoresis has been used for separating mixtures of molecules based on their different rates of travel in electric fields. Generally, electrophoresis refers to the movement of suspended or dissolved molecules through a fluid or gel under the action of an electromotive force applied to one or more electrodes or electrically conductive members in contact with the fluid or gel. Some known modes of electrophoretic separation include separating molecules based, at least in part, on differences in their mobilities in a buffer solution (commonly referred to as zone electrophoresis), in a gel or polymer solution (commonly referred to as gel electrophoresis), or in a potential of hydrogen (pH) gradient (commonly referred to as isoelectric focusing). Even though capillary electrophoresis techniques are effective and widely used in the industry to study biomolecule purity and charge heterogeneity, it does not allow selective detection of various species or allow differentiation of product and process impurities. Accordingly, additional methods of monitoring mAb preparations and formulations for detecting host cell protein impurities are needed.

### SUMMARY OF THE INVENTION

In one aspect, the present invention provides a method for detecting protein contaminants of interest in an antibody preparation sample, in which the method includes: separating protein components of a sample by a physical parameter in one or more capillaries using capillary electrophoresis; immobilizing the protein components of the sample within the one or more capillaries; contacting the protein components within the one or more capillaries with one or more primary antibodies that specifically bind to a protein contaminant of interest; and detecting the binding of the one or more primary antibodies, thereby detecting and quantifying protein contaminants of interest in the antibody preparation sample.

In some embodiments, the method further comprises discriminating between variants of the protein contaminant of interest in an antibody preparation sample by the physical parameter.

In various embodiments of the method, the one or more capillaries comprise a separation matrix.

In various embodiments of the method, the separation matrix comprises carrier ampholytes.

In various embodiments of the method, the physical parameter comprises charge.

In various embodiments of the method, the separation matrix comprises a sieving matrix configured to separate proteins by molecular weight.

In various embodiments of the method, the physical parameter comprises molecular weight.

In various embodiments of the method, the one or more primary antibodies are labeled with a detectable label, and detecting the binding of the one or more primary antibodies comprises detecting the detectable label.

In some embodiments, detecting the binding of the one or more primary antibodies comprises: contacting the one or more primary antibodies with a secondary antibody that specifically binds at least one of the one or more primary antibodies, and wherein the secondary antibody has a detectable label; and detecting the detectable label.

In some embodiments, the method further comprises detecting and/or discriminating between charge or size variants of the protein contaminants of interest.

In some embodiments, the method further comprises determining a relative or absolute amount of the protein contaminants of interest.

In various embodiments of the method, the detectable label comprises a chemiluminescent label, a fluorescent label or a bioluminescent label.

In various embodiments of the method, the sample includes an internal standard.

In some embodiments, immobilizing comprises photo-immobilizing, chemically immobilizing, or thermally immobilizing.

In various embodiments of the method, the one or more primary antibodies comprise polyclonal antibodies.

In various embodiments of the method, the one or more primary antibodies comprise monoclonal antibodies.

In various embodiments of the method, protein contaminants of interest comprise of PLBD2, CTSD, TIMP1, Acid Ceramidase (ASAH1), Lysosomal Acid Lipase (LAL),Annexin, Cathepsin B, Antileukoproteinase (ALP), or a fragment thereof.

In another aspect, the present invention provides a method for detecting and/or discriminating between protein contaminants of interest in an antibody preparation sample by a physical parameter, in which the method includes: separating protein components of a sample by a physical parameter in one or more capillaries using capillary electrophoresis; immobilizing the protein components of the sample within the one or more capillaries; contacting the protein components within the one or more capillaries with a first primary antibody that specifically binds to a first protein contaminants of interest; detecting the binding of the a first primary antibody, thereby detecting the first antibody of interest; contacting the protein components within the one or more capillaries with a second primary antibody that specifically binds to a second protein contaminant of interest; and detecting the binding of the second primary antibody, thereby detecting the protein contaminants of interest and discriminating between the protein contaminants of interest in a sample.

In some embodiments, the method further comprises contacting the protein components within the one or more capillaries with a third primary antibody that specifically binds to a third protein contaminant of interest; detecting the binding of the third primary antibody, thereby detecting the third protein contaminant of interest.

In some embodiments, the method further comprises contacting the protein components within the one or more capillaries with one or more additional primary antibodies that specifically binds to one or more additional protein contaminants of interest; detecting the binding of the one or more additional primary antibodies, thereby detecting the one or more additional protein contaminants of interest.

In some embodiments, the method further comprises discriminating between variants of the protein contaminants of interest in an antibody preparation sample by the physical parameter.

In various embodiments of the method, the one or more capillaries comprise a separation matrix.

In various embodiments of the method, the separation matrix comprises carrier ampholytes.

In various embodiments of the method, the physical parameter comprises charge.

In various embodiments of the method, the separation matrix comprises a sieving matrix configured to separate proteins by molecular weight.

In various embodiments of the method, the physical parameter comprises molecular weight.

In various embodiments of the method, the primary antibodies are labeled with a detectable label, and wherein detecting the binding of the primary antibodies comprises detecting the detectable label.

In some embodiments, detecting the binding of the primary antibodies comprises: contacting the primary antibodies with a secondary antibody that specifically binds the primary antibodies, wherein the secondary antibody has a detectable label; and detecting the detectable label.

In some embodiments, the method further comprises determining a relative or absolute amount of one or more of the protein contaminants of interest.

In various embodiments of the method, the detectable label comprises a chemiluminescent label, a fluorescent label or a bioluminescent label.

In various embodiments of the method, the sample includes an internal standard.

In various embodiments of the method, the one or more primary antibodies comprise polyclonal antibodies.

In various embodiments of the method, the one or more primary antibodies comprise monoclonal antibodies.

In various embodiments of the method, the immobilizing comprises photo-immobilizing, chemically immobilizing, or thermally immobilizing.

In various embodiments of the method, the protein contaminants of interest comprise of PLBD2, CTSD, TIMP1, Acid Ceramidase (ASAH1), Lysosomal Acid Lipase (LAL),Annexin, Cathepsin B, Antileukoproteinase (ALP), or a fragment thereof.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### DESCRIPTION OF THE FIGURES

FIG. 1A is a digital image of an SDS-PAGE and a western blot showing the forms of a preparation of the polypeptide PLBD2.

FIG. 1B is a diagram showing the proposed forms of PLBD2.

FIG. 2 is a set of digital images of Western blots using selected anti-PLBD2 antibody preparations. Mice were immunized using recombinant PLBD2 or HIC strip to generate anti-PLBD2 mAbs. Hybridomas were screened for specificity by western blot and 10 were selected for purification and biotinylation. Mature PLBD2 protein (−42 kDa) was not detected in any of the hybridomas. Antibodies targeting the N-terminus were identified.

FIG. 3 is a bar graph showing the activity of anti-PLBD2 antibodies. From these studies, mAb09 coating and biotinylated goat pAb detection were selected for the final sandwich ELISA format.

FIG. 4 is a schematic representation of a sandwich ELISA using selected anti-PLBD2 antibodies.

FIG. 5 is a standard curve generated for a selected pair of anti-PLBD2 antibodies.

FIG. 6 is a of an exemplary work flow for the separation and detection of polypeptide contaminants by capillary electrophoresis using approximate molecular weight.

FIG. 7 shows a set of figures demonstrating a concentration dependent analysis of PLBD2 in reducing and non-reducing conditions. This result shows the quantitation of PLBD2 in an antibody sample.

FIG. 8 is a diagram of an exemplary work flow for the separation and detection of polypeptides by capillary electrophoresis using charge.

FIG. 9 shows the results of an imaged cIEF-Western (icIEF-Western) Charge Assay. PLBD2 is detected using the anti-PLBD2 pAb. PLBD2 is absent in the C2P2 process and inclusion of the sample confirms that the CE-western is specifically picking up the PLBD2 peaks in the 5-6 region.

FIG. 10 shows the results of an imaged cIEF-Western (icIEF-Western) Charge Assay. The native PLBD2 can be seen in the pH range of 5-6 in the figure on the right. This was detected from the mAb process, demonstrating the ability of this method to selectively detect the PLBD2 from the process samples. In this charge mode, a specific polyclonal and or monoclonal antibody to PLBD2 can be used to detect the impurity in the process sample.

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.)

Although any methods and materials similar or equivalent to those described herein can be used in the practice or testing of the present invention, the preferred methods and materials are now described. All patents, applications and non-patent publications mentioned in this specification are incorporated herein by reference in their entireties.

Abbreviations Used Herein

mAb: Monoclonal antibody

biAb: Bispecific antibody

CE: Capillary Electrophoresis

SDS: Sodium dodecyl sulfate

icIEF: Imaged CIEF

icIEF-western; Charged based CE-Western

IEC: Ion exchange chromatography

QC: Quality control

HRP: Horse radish peroxidase

HCPs: Host Cell Proteins

Definitions

The term “antibody”, as used herein, is intended to refer to immunoglobulin molecules included of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is included of a heavy chain variable region (“HCVR” or “V_{H}”) and a heavy chain constant region (included of domains C_{H}1, C_{H}2 and C_{H}3). In various embodiments, the heavy chain may be an IgG isotype. In some cases, the heavy chain is selected from IgG1, IgG2, IgG3 or IgG4. In some embodiments, the heavy chain is of isotype IgG1 or IgG4, optionally including a chimeric hinge region of isotype IgG1/IgG2 or IgG4/IgG2. Each light chain is included of a light chain variable region (“LCVR” or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. For a review on antibody structure, see Lefranc et al., IMGT unique numbering for immunoglobulin and T cell receptor variable domains and Ig superfamily V-like domains, 27(1) Dev. Comp. Immunol. 55-77 (2003); and M. Potter, Structural correlates of immunoglobulin diversity, 2(1) Surv. Immunol. Res. 27-42 (1983).

The term antibody also encompasses a “bispecific antibody”, which includes a heterotetrameric immunoglobulin that can bind to more than one epitope. One half of the bispecific antibody, which includes a single heavy chain and a single light chain and six CDRs, binds to one antigen or epitope, and the other half of the antibody binds to a different antigen or epitope. In some cases, the bispecific antibody can bind the same antigen, but at different epitopes or non-overlapping epitopes. In some cases, both halves of the bispecific antibody have identical light chains while retaining dual specificity. Bispecific antibodies are described generally in U.S. Patent App. Pub. No. 2010/0331527(Dec. 30, 2010).

The term “antigen-binding portion” of an antibody (or “antibody fragment”), refers to one or more fragments of an antibody that retain the ability to specifically bind to an antigen. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et at. (1993) 90 PNAS U.S.A. 6444-6448; and Poljak et at. (1994) 2 Structure 1121-1123).

Moreover, antibodies and antigen-binding fragments thereof can be obtained using standard recombinant DNA techniques commonly known in the art (see Sambrook et al., 1989).

The term “human antibody”, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “sample,” as used herein, refers to a mixture of molecules that includes at least one polypeptide of interest, such as a monoclonal antibody, a bispecific antibody and/or one or more host cells protein (HCP) contaminants, that is subjected to manipulation in accordance with the methods of the invention, including, for example, separating, analyzing, extracting, concentrating or profiling.

The terms “analysis” or “analyzing,” as used herein, are used interchangeably and refer to any of the various methods of separating, detecting, isolating, purifying, solubilizing, detecting and/or characterizing molecules of interest (e.g., polypeptides, such as antibodies and HCP contaminants) in biopharmaceutical preparations, such as antibody preparations.

“Chromatography,” as used herein, refers to the process of separating a mixture, for example a mixture containing peptides, proteins, polypeptides and/or antibodies, such as monoclonal antibodies. It involves passing a mixture through a stationary phase, which separates molecules of interest from other molecules in the mixture and allows one or more molecules of interest to be isolated. In the method disclosed herein chromatography refers to capillary electrophoresis, including size based capillary electrophoresis and isoelectric focusing or charged based capillary electrophoresis.

“Contacting,” as used herein, includes bringing together at least two substances in solution or solid phase, for example contacting a sample with an antibody, such as an antibody that specifically binds to a molecule of interest, such as a HCP contaminant.

The term “isolated,” as used herein, refers to a biological component (such as an antibody, for example a monoclonal antibody) that has been substantially separated, produced apart from, or purified away from other biological components in the cell of the organism in which the component naturally occurs or is transgenically expressed, that is, other chromosomal and extrachromosomal DNA and RNA, proteins, lipids, and metabolites. Nucleic acids, peptides, proteins, lipids and metabolites which have been “isolated” thus include nucleic acids, peptides, proteins, lipids, and metabolites purified by standard or non-standard purification methods. The term also embraces nucleic acids, peptides, proteins, lipids, and metabolites prepared by recombinant expression in a host cell as well as chemically synthesized peptides, lipids, metabolites, and nucleic acids.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr. In one embodiment a peptide is an antibody or fragment or part thereof, for example, any of the fragments or antibody chains listed above. In some embodiments, the peptide may be post-translationally modified. In another embodiment, a peptide is an HCP contaminant.

“Detect” and “detection” have their standard meaning, and are intended to encompass detection including the presence or absence, measurement, and/or characterization of an protein of interest, such as a contaminant polypeptide, for example an HCP.

As used herein, the terms “protein of interest” and/or “target protein of interest” refer to any protein to be separated and/or detected with the methods, provided herein. Suitable protein of interests include contaminating proteins in antibody preparations, such as HCPs.

As used herein, the terms “standard” and/or “internal standard” refer to a well-characterized substance of known amount and/or identity (e.g., known molecular weight, electrophoretic mobility profile) that can be added to a sample and both the standard and the molecules in the sample, on the basis of molecular weight or isoelectric point by electrophoresis). A comparison of the standard then provides a quantitative or semi-quantitative measure of the amount of analyte, such as a contaminant protein present in the sample, for example, an HCP.

General Description

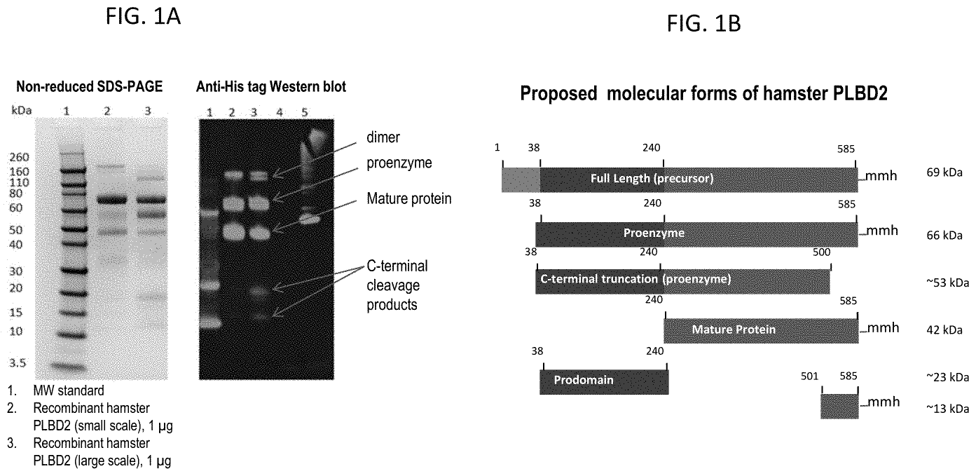

Characterization of contaminating host cell protein variants is important in order to identify their potential impact on the purification of potential or realized therapeutic antibodies. In addition to the characterization of mAbs, understanding the nature of protein contaminants is another important factor in the development of mAb therapeutics. For example, control of residual protein A, HCP, residual DNA and other potential culture or purification residues are typically part of the drug substance specification. In addition, such control provides valuable information on process consistency and performance. Thus, disclosed herein are size and/or charge based detection methods for Host Cell Proteins (HCPs), for example using antibodies, such as monoclonal or polyclonal antibodies specific for the HCPs, e.g. contaminating proteins of interest. The disclosed methods allow for the detection and visualization of problematic HCPs and their various species in process samples (see for example FIGS. 1A and 1B, which show heterogeneity in the hamster protein PLBD2, a common contaminant in samples purified from CHO cells). As used herein, PLBD2 refers to the gene or the gene product, e.g. the PLBL2 protein produced by the PLBD2 gene. Thus, PLBD2 can refer to the gene or the gene product, which is synonymous with the PLBL2 protein. These methods allow for the ability to detect and show the various species of a given HCP impurity at low ppm levels. Thus, aspects of this disclosure include a method for detecting protein contaminants of interest in a monoclonal antibody preparation sample. The ability to discriminate between more contaminating host cell proteins of interest or fragments thereof, in a biological sample, is becoming increasingly important as the activity of the protein and/or fragments may have differing effects on the activity of the active agent, such as a therapeutic antibody. Thus, methods are needed to characterize potential therapeutic mAbs and potential contaminants of mAb preparations. The methods disclosed herein meet those needs.

Disclosed herein is a method for detecting and/or discriminating between variants of contaminating host cell proteins in a biological sample, such as a monoclonal antibody (mAb) preparation by a physical parameter, such as the molecular weight or the isoelectric point of the contaminating host cell protein. The disclosed methods can be used in QC evaluation of antibody preparations. In embodiments of the method, a sample that includes a contaminating host cell protein or multiple contaminating host cell proteins of interest is resolved or separated by using capillary electrophoresis, for example on one or more capillaries of a CE-system. In certain embodiments, the sample is resolved or separated by molecular weight. Resolution by molecular weight allows for the determination of what fragments or species of contaminating host cell proteins are present in the sample. In certain embodiments, the sample is resolved or separated by charge, for example by isoelectric focusing. Separation of the contaminating host cell proteins by charge has the added benefit of being able to determine the homogeneity of the contaminating host cell proteins, for example, changes in surface charge of the contaminating host cell proteins that may not be easily seen in separation by molecular weight. In certain embodiments, the sample is resolved or separated within a single capillary. In certain embodiments, the sample is resolved or separated within multiple capillaries, for example in parallel.

Once the protein components have been resolved or separated in the one or more capillaries, the protein components, for example the contaminating host cell proteins of interest, are immobilized within the capillary so that the relative position of the contaminating host cell proteins of interest in the one or more capillaries is maintained. In embodiments, the contaminating host cell protein of interest is detected by contacting the protein components within the one or more capillaries, including the contaminating host cell protein of interest, with one or more primary antibodies that specifically bind to the contaminating host cell protein of interest or fragments thereof to detect the presence of the contaminating host cell protein or fragments thereof. In embodiments, the method includes detecting the binding of the one or more primary antibodies, for example because its mobility in the capillary is impaired by the immobilization of the or fragments thereof. Detection of the binding of the primary antibody, for example along the length of a capillary, allows for the detecting of and/or discrimination between size and/or change variants of the contaminating host cell proteins of interest or fragments thereof in the sample, depending on weather the sample was subjected to separation by mass or charge, respectively. By way of example with respect to separation by molecular weight, the smaller the fragment the further within a capillary it would be expected to travel. In embodiments, the sample may contain multiple, such as at least 2, at least 3, at least 4, at least 5 or more contaminating host cell proteins of interest or fragments thereof, each of which can be detected using a primary antibody that specifically binds to the individual contaminating host cell protein(s) of interest or fragments thereof. In some embodiments, the method further includes determining a relative or absolute amount of the variants of the contaminating host cell proteins of interest in a sample, for example by measurement of peak height or area, which corresponds to the amount of labeled primary antibody detected and therefore how much contaminating host cell protein or fragments thereof is available to bind the labeled primary antibody. In some embodiments, the contaminating host cell proteins of interest comprises one or more of PLBD2, CTSD, TIMP1, Acid Ceramidase (ASAH1), Lysosomal Acid Lipase (LAL),Annexin, Cathepsin B, Antileukoproteinase (ALP), or a fragment thereof. In some embodiments, the protein contaminants of interest comprise PLBD2. In some embodiments, the sample includes one or more internal standards, for example a ladder of molecular weight standards, a ladder of isoelectric point standards, or even a standard used as a baseline or benchmark for determining the amount of a contaminating host cell protein of interest or a fragment thereof, in the sample. In some embodiments, the method includes detecting and/or discriminating between charge or size variants of the protein contaminants of interest. In some embodiments, a relative or absolute amount of the protein contaminants of interest can be determined. In various embodiments of the method, the one or more primary antibodies comprise polyclonal antibodies In various embodiments of the method, the one or more primary antibodies comprise monoclonal antibodies.

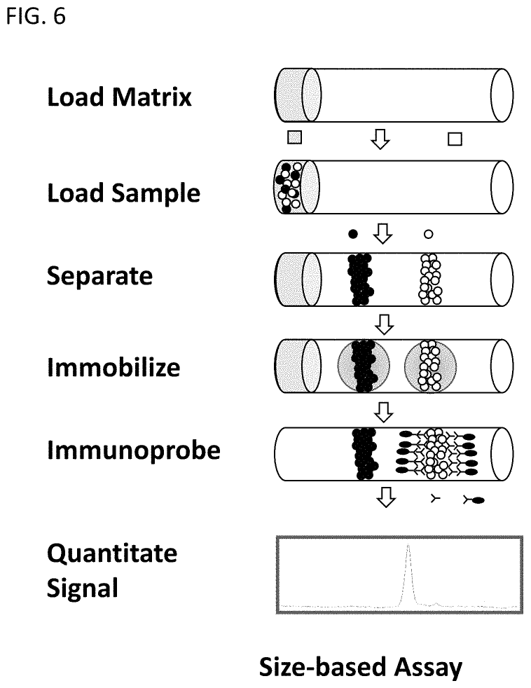

In embodiments, the method includes separating protein components of a sample with two or more size variants of contaminating host cell proteins of interest, such as 2, 3, 4, 5, 6, 7, 8, 9, 10 or even more, contaminating host cell protein(s) of interest, by molecular weight in one or more capillaries using capillary electrophoresis. An example flow is given in FIG. 6. In embodiments, the method includes immobilizing the protein components of the sample within the one or more capillaries. In embodiments, the method includes contacting the protein components within the one or more capillaries with a first primary antibody that specifically binds to a first monoclonal antibody of interest. In embodiments, the method includes detecting the binding of the first primary antibody, thereby detecting the first monoclonal antibody of interest. In some embodiments, a molecular weight based profile or fingerprint of the contaminating host cell protein can be created, for example of the contaminating host cell protein of interest alone for comparison with a molecular weight based profile or fingerprint of the contaminating host cell proteins in the mixture. This comparison can then be used to determine if the contaminating host cell protein of interest changes in the mixture, for example over time or across preparation. This can be done to optimize the conditions for the preparation, for example to minimize the effects or activity of the contaminating host cell proteins that may be present in the preparation of a given therapeutic mAb. This profile or fingerprint comparison can be done for any or all of the contaminating host cell proteins of interest in the mixture. In embodiments, the method includes contacting the protein components within the one or more capillaries with a second primary antibody that specifically binds to a second monoclonal antibody of interest. In embodiments, the method includes detecting the binding of a second primary antibody, thereby detecting the second monoclonal antibody of interest and discriminating between the contaminating host cell proteins in a sample. This can be continued for multiple different contaminating host cell proteins in the sample. For example, in embodiments, the method can include contacting the protein components within the one or more capillaries with a third primary antibody that specifically binds to a third contaminating host cell protein of interest and detecting the binding of the third primary antibody, thereby detecting the contaminating host cell protein of interest. In additional embodiments, the method can include contacting the protein components within the one or more capillaries with one or more additional primary antibodies, for example a 4^{th}, 5^{th}, 6^{th}, 7^{th}, and so on, primary antibody, that specifically binds to one or more additional contaminating host cell protein(s) of interest, for example a 4^{th}, 5^{th}, 6^{th}, 7^{th}, and so on additional contaminating host cell protein(s) of interest, and detecting the binding of the one or more additional primary antibodies, thereby detecting the contaminating host cell protein(s) of interest. In embodiments, the sample is split into multiple capillaries and each of these capillaries are contacted with a different primary antibody or antibodies, and detected. The signals obtained can be later combined. In certain embodiments, the detection can take place in a single capillary, for example in multiplex.

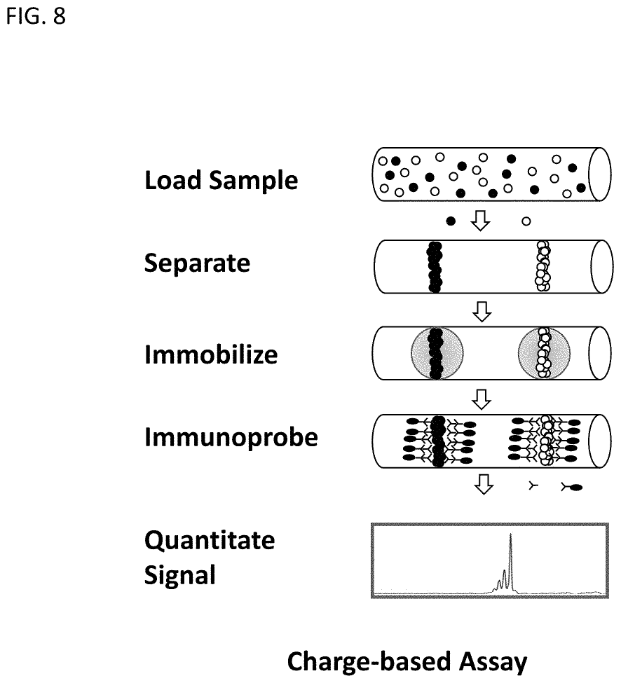

In embodiments, the method includes separating protein components of a sample with two or more charge variants of contaminating host cell protein of interest, such as 2, 3, 4, 5, 6, 7, 8, 9, 10 or even more, contaminating host cell protein(s) of interest, by charge in one or more capillaries using capillary electrophoresis, for example by isoelectric focusing. In embodiments, the method includes immobilizing the protein components of the sample within the one or more capillaries. An example flow is given in FIG. 8. In embodiments, the method includes contacting the protein components within the one or more capillaries with a first primary antibody that specifically binds to a first monoclonal antibody of interest. In embodiments, the method includes detecting the binding of the first primary antibody, thereby detecting the first monoclonal antibody of interest. In some embodiments, a charge based profile or fingerprint of the contaminating host cell protein can be created, for example of the contaminating host cell protein of interest alone for comparison with a charge based profile or fingerprint of the contaminating host cell proteins in the mixture. This comparison can then be used to determine if the contaminating host cell protein of interest changes in the mixture, for example over time or across preparation. This can be done to optimize the conditions for the preparation, for example to minimize the effects or activity of the contaminating host cell protein that may be present in the preparation of a given therapeutic mAb. This profile or fingerprint comparison can be done for any or all of the contaminating host cell proteins of interest in the mixture. In embodiments, the method includes contacting the protein components within the one or more capillaries with a second primary antibody that specifically binds to a second monoclonal antibody of interest. In embodiments, the method includes detecting the binding of a second primary antibody, thereby detecting the second monoclonal antibody of interest and discriminating between the contaminating host cell proteins in a sample. This can be continued for multiple different contaminating host cell protein in the sample. For example, in embodiments, the method can include contacting the protein components within the one or more capillaries with a third primary antibody that specifically binds to a third contaminating host cell protein of interest and detecting the binding of the third primary antibody, thereby detecting the contaminating host cell protein of interest. In additional embodiments, the method can include contacting the protein components within the one or more capillaries with one or more additional primary antibodies, for example a 4^{th}, 5^{th}, 6^{th}, 7^{th}, and so on, primary antibody, that specifically binds to one or more additional contaminating host cell protein(s) of interest, for example a 4^{th}, 5^{th}, 6^{th}, 7^{th}, and so on additional contaminating host cell protein(s) of interest, and detecting the binding of the one or more additional primary antibodies, thereby detecting the contaminating host cell protein(s) of interest. In embodiments, the sample is split into multiple capillaries and each of these capillaries are contacted with a different primary antibody or antibodies and detected. The signals obtained can be later combined. In certain embodiments, the detection can take place in a single capillary, for example in multiplex.

Samples for use in the disclosed methods can be heterogeneous, containing a variety of components, i.e., different proteins. Alternatively, the sample can be homogenous, containing one component or essentially one component of multiple charge or molecular weight species. Pre-analysis processing may be performed on the sample prior to detecting the protein of interest, such as a contaminating protein. For example, the sample can be subjected to a lysing step, denaturation step, heating step, purification step, precipitation step, immunoprecipitation step, column chromatography step, centrifugation, etc. In some embodiments, the separation of the sample and immobilization may be performed on native substrates. In other embodiments, the sample may be subjected to denaturation, for example, heat and/or contact with a denaturizing agent. Denaturizing agents are known in the art. In some embodiments, the sample may be subjected to non-reducing conditions. In some embodiments, the sample may be subjected to reducing conditions, for example contacted with one or more reducing agents. Reducing agents are knowns in the art.

In some embodiments, the primary antibodies are labeled with a detectable label and detecting the binding of the one or more primary antibodies comprises detecting the detectable label. In some embodiments, detecting the binding of the one or more primary antibodies includes contacting the one or more primary antibodies with a secondary antibody that specifically binds at least one of the one or more primary antibodies and detecting the binding of the secondary antibody. In embodiments, the secondary antibody has a detectable label and the detectable label is detected.

In some embodiments, the primary antibodies and/or secondary antibodies include one or more detectable labels. In some embodiments, the detectable label comprises a chemiluminescent label, a fluorescent label or bioluminescent label. In some embodiments, the detectable label includes a chemiluminescent label. The chemiluminescent label can include any entity that provides a light signal and that can be used in accordance with the methods disclosed herein. A variety of such chemiluminescent labels are known in the art, see for example, e.g., U.S. Pat. Nos. 6,689,576, 6,395,503, 6,087,188, 6,287,767, 6,165,800, and 6,126,870. Suitable labels include enzymes capable of reacting with a chemiluminescent substrate in such a way that photon emission by chemiluminescence is induced. Such enzymes induce chemiluminescence in other molecules through enzymatic activity. Such enzymes may include peroxidase, such as horse radish peroxidase (HRP), beta-galactosidase, phosphatase, or others for which a chemiluminescent substrate is available. In some embodiments, the chemiluminescent label can be selected from any of a variety of classes of luminol label, an isoluminol label, etc. In some embodiments, the primary antibodies include chemiluminescent labeled antibodies. Chemiluminescent substrates are well known in the art, such as Galacton substrate available from Applied Biosystems of Foster City, Calif. or SuperSignal West Femto Maximum Sensitivity substrate available from Pierce Biotechnology, Inc. of Rockford, Ill. or other suitable substrates.

In some embodiments, the detectable label includes a bioluminescent compound. Bioluminescence is a type of chemiluminescence found in biological systems in which a catalytic protein increases the efficiency of the chemiluminescent reaction. The presence of a bioluminescent compound is determined by detecting the presence of luminescence. Suitable bioluminescent compounds include, but are not limited to luciferin, luciferase and aequorin.

In some embodiments, the detectable label includes a fluorescent label, such as a fluorescent dye. A fluorescent dye can include any entity that provides a fluorescent signal and that can be used in accordance with the methods and devices described herein. Typically, the fluorescent dye includes a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, non-limiting examples include xanthenes, rhodamines, fluoresceins, cyanines, phthalocyanines, squaraines, bodipy dyes, coumarins, oxazines, and carbopyronines. In some embodiments, for example, where primary and/or secondary antibodies contain fluorophores, such as fluorescent dyes, their fluorescence is detected by exciting them with an appropriate light source, and monitoring their fluorescence by a detector sensitive to their characteristic fluorescence emission wavelength. In some embodiments, the primary antibodies include fluorescent dye labeled antibodies.

In embodiments, using two or more different primary or secondary antibodies, which bind to or interact with different proteins of interests, such as different contaminant proteins of interest, different types of proteins of interest can be detected simultaneously, for example in multiplex within the same or a single capillary, for example using different or even the same detectable label. In some embodiments, multiple primary and/or secondary antibodies can be used with multiple substrates to provide color-multiplexing. For example, the different chemiluminescent substrates used would be selected such that they emit photons of differing color. Selective detection of different colors can be accomplished by using a diffraction grating, prism, series of colored filters, or other means.

In embodiments, the capillary may include a separation matrix, which can be added in an automated fashion by the apparatus and/or system. In some embodiments, the sample is loaded onto a stacker matrix prior to separation. The separation matrix, in one embodiment, is a size separation matrix, and has similar or substantially the same properties of a polymeric gel, used in conventional electrophoresis techniques. Capillary electrophoresis in the separation matrix is analogous to separation in a polymeric gel, such as a polyacrylamide gel or an agarose gel, where molecules are separated on the basis of the size of the molecules in the sample, by providing a porous passageway through which the molecules can travel. The separation matrix permits the separation of molecules by molecular size because larger molecules will travel more slowly through the matrix than smaller molecules. In some embodiments, the one or more capillaries comprise a separation matrix. In some embodiments, the sample containing a protein of interest is separated or resolved based on molecular weight. In some embodiments, the separation matrix comprises a sieving matrix configured to separate proteins by molecular weight. In some embodiments, protein components of a sample are separated by molecular weight and the method is a method of detecting and/or discriminating between size variants of a monoclonal antibody of interest. In some embodiments, protein components of a sample are separated by molecular weight and the method is a method of detecting and/or discriminating between size variants of a contaminating protein of interest.

A wide variety of solid phase substrates are known in the art, for example gels, such as polyacrylamide gel. In some embodiments, resolving one or more proteins of interest includes electrophoresis of a sample in a polymeric gel. Electrophoresis in a polymeric gel, such as a polyacrylamide gel or an agarose gel, separates molecules on the basis of the molecule's size. A polymeric gel provides a porous passageway through which the molecules can travel. Polymeric gels permit the separation of molecules by molecular size because larger molecules will travel more slowly through the gel than smaller molecules.

In some embodiments, the sample containing a protein of interest is separated or resolved based on the charge of the components of the sample. In some embodiments, protein components of a sample are separated by charge and the method is a method of detecting and/or discriminating between charge variants of a monoclonal antibody of interest. In some embodiments, protein components of a sample are separated by charge and the method is a method of detecting and/or discriminating between charge variants of a contaminating protein of interest. In some embodiments, the separation matrix comprises carrier ampholytes. In some embodiments, separating a sample by charge includes isoelectric focusing (IEF) of a sample. For example, in an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches a spot where its net charge is zero (i.e., its isoelectric point) and it is thereafter immobilized in the electric field. Thus, this electrophoresis procedure separates molecules according to their different isoelectric points.

In some embodiments, for example, when resolving is by isoelectric focusing, an ampholyte reagent can be loaded into one or more capillaries of a capillary electrophoresis device. An ampholyte reagent is a mixture of molecules having a range of different isoelectric points. Typical ampholyte reagents are Pharmalyte™ and Ampholine™ available from Amersham Biosciences of Buckinghamshire, England.

In embodiments, once the separation is complete, the components of the separated sample (e.g., including the proteins of interest, such as a contaminating protein of interest, are immobilized to a wall(s) of the one or more capillaries using any suitable method including but not limited to chemical, photochemical, and heat treatment. In some embodiments, the components of the separated sample are immobilized in one or more capillaries of a CE-system after the molecules have been separated by electrophoresis, for example by size or charge. In some embodiments, the immobilizing comprises photo-immobilizing, chemically immobilizing, or thermally immobilizing. The immobilization can be via covalent bonds or non-covalent means such as by hydrophobic or ionic interaction. In certain embodiments, the protein(s) of interest are immobilized using one or more reactive moieties. The reactive moiety can include any reactive group that is capable of forming a covalent linkage with a corresponding reactive group of individual molecules of the sample. Thus, the reactive moiety can include any reactive group known in the art, so long as it is compatible with the methods disclosed herein. In some embodiments, the reactive moiety includes a reactive group that is capable of forming a covalent linkage with a corresponding reactive group of an protein of interest, such as a contaminating protein of interest.

The reactive moiety can be attached directly, or indirectly to the capillary. In some embodiments, the reactive moiety can be supplied in solution or suspension, and may form bridges between the wall of the capillary and the molecules in the sample upon activation. For example, in one embodiment, immobilization occurs by subjecting the separated sample and the capillaries to ultraviolet (UV) light, which serves to immobilize the protein of interest(s) (if present in the sample) and molecules in the sample to the walls of the capillary. The immobilization can be via covalent bonds or non-covalent means such as by hydrophobic or ionic interaction. In another embodiment, a reactive moiety can be used to covalently immobilize the resolved protein of interest or proteins of interest in the capillary. The reactive moiety can be attached directly or indirectly to the capillary (e.g., on the wall(s) of the capillary tube). In some embodiments, the reactive moiety can be supplied in solution or suspension, and can be configured to form bridges between the wall of the capillary and the molecules in the sample upon activation. The reactive moiety can line the capillary or can be present on a linear or cross-linked polymer in the capillary, which may or may not be linked to the wall of the capillary before and/or after activation. The reactive moiety can be and/or can include any reactive group that is capable of forming a covalent linkage with a corresponding reactive group of individual molecules of the sample such as, for example, those described above.

In some embodiments, the reactive moiety includes a functional group that can be converted to a functionality that adheres to a protein of interest via hydrophobic interactions, ionic interactions, hydrogen bonding etc. In some embodiments, such reactive moieties are activated with UV light, a laser, temperature, or any other source of energy in order to immobilize the protein of interest onto the surfaces of the capillary and/or onto the surfaces of particles attached to the surfaces of the capillary. In some embodiments, the surfaces of the capillary are functionalized with thermally responsive polymers that enable changes in hydrophobicity of the surfaces upon changing the temperature. In some embodiments, the proteins of interest are immobilized on such surfaces by increasing hydrophobicity of a temperature responding polymer when a certain temperature is reached within the capillary.

A wide variety of reactive moieties suitable for covalently linking two molecules together are known in the art. For example, the reactive moiety can bind to carbon-hydrogen (C—H) bonds of proteins. Since many separation media also contain components with C—H bonds, chemistries that react with sulfhydryl (S—H) groups may be advantageous in that S—H groups are found uniquely on proteins relative to most separation media components. Suitable reactive moieties include, but are not limited to, photoreactive groups, chemical reactive groups, and thermoreactive groups. Photoimmobilization in the capillary system can be accomplished by the activation of one or more photoreactive groups. A photoreactive group includes one or more latent photoreactive groups that upon activation by an external energy source, forms a covalent bond with other molecules. See, e.g., U.S. Pat. Nos. 5,002,582 and 6,254,634. The photoreactive groups generate active species such as free radicals and particularly nitrenes, carbenes, and excited states of ketones upon absorption of electromagnetic energy. The photoreactive groups can be chosen that are responsive to various portions of the electromagnetic spectrum, such as those responsive to ultraviolet, infrared and visible portions of the spectrum. For example, upon exposure to a light source, the photoreactive group can be activated to form a covalent bond with an adjacent molecule. Suitable photoreactive groups include, but are not limited to, aryl ketones, azides, diazos, diazirines, and quinones. In some embodiments, the resolved proteins of interest of the sample are immobilized in the capillary of a CE-system by isoelectric focusing.

Detecting a detectable label can be by any method known in the art, so long as it is compatible with the methods described herein. Label detection can be performed by monitoring a signal using conventional methods and instruments, non-limiting examples include, a photodetector, an array of photodetectors, a charged coupled device (CCD) array, etc. Typically, detecting the detectable label includes imaging the capillary. In some embodiments, the entire length of the capillary can be imaged. Alternatively, a distinct part or portion of the capillary can be imaged.

Variations of order of the steps of the methods described herein will readily occur to those skilled in the art. For example, the sample can be separated and then the protein of interest(s) immobilized at their resolved locations in the capillary, prior to contacting the protein of interest(s) with the primary antibodies. In some embodiments, primary antibodies are contacted with the protein of interest(s) to form a complex and then the complex is resolved in the capillary of a CE-system. In some embodiments, the primary antibodies could be preloaded into the sample and thereafter loaded into the system. As another example, the resolving step, such as isoelectric focusing, can be applied after the chemiluminescent reagents are supplied.

In some embodiments, sample includes an internal standard. Internal standards serve to calibrate the separation with respect to isoelectric point or molecular weight. Internal standards for IEF are well known in the art, for example see, Shimura, K., Kamiya, K., Matsumoto, H., and K. Kasai (2002) Fluorescence-Labeled Peptide pI Markers for Capillary Isoelectric Focusing, Analytical Chemistry v74: 1046-1053, and U.S. Pat. No. 5,866,683. Standards to be detected by fluorescence could be illuminated either before or after chemiluminescence, but generally not at the same time as chemiluminescence. In some embodiments, the protein of interest and standards are detected by fluorescence. The protein of interest and standards can each be labeled with fluorescent dyes that are each detectable at discrete emission wavelengths, such that the protein of interest and standards are independently detectable.

In some embodiments, an internal standard can be a purified form of the protein of interest itself, which is generally made distinguishable from the protein of interest in some way. Methods of obtaining a purified form of the protein of interest can include, but are not limited to, purification from nature, purification from organisms grown in the laboratory (e.g., via chemical synthesis), and/or the like. The distinguishing characteristic of an internal standard can be any suitable change that can include, but is not limited to, dye labeling, radiolabeling, or modifying the mobility of the standard during the electrophoretic separation so that it is separated from the protein of interest. For example, a standard can contain a modification of the protein of interest that changes the charge, mass, and/or length (e.g., via deletion, fusion, and/or chemical modification) of the standard relative to the protein of interest. Thus, the protein of interest and the internal standard can each be labeled with fluorescent dyes that are each detectable at discrete emission wavelengths, thereby allowing the protein of interest and the standard to be independently detectable. In some instances, an internal standard is different from the protein of interest but behaves in a way similar to or the same as the protein of interest, enabling relevant comparative measurements. In some embodiments, a standard that is suitable for use can be any of those described in U.S. Patent Application Publication No. 2007/0062813, the disclosure of which is incorporated herein by reference in its entirety.

As will be appreciated by those in the art, virtually any method of loading the sample in the capillary may be performed. For example, the sample can be loaded into one end of the capillary. In some embodiments, the sample is loaded into one end of the capillary by hydrodynamic flow. For example, in embodiments wherein the fluid path is a capillary, the sample can be loaded into one end of the capillary by hydrodynamic flow, such that the capillary is used as a micropipette. In some embodiments, the sample can be loaded into the capillary by electrophoresis, for example, when the capillary is gel filled and therefore more resistant to hydrodynamic flow.

The capillary can include any structure that allows liquid or dissolved molecules to flow. Thus, the capillary can include any structure known in the art, so long as it is compatible with the methods. In some embodiments, the capillary is a bore or channel through which a liquid or dissolved molecule can flow. In some embodiments, the capillary is a passage in a permeable material in which liquids or dissolved molecules can flow.

The capillary includes any material that allows the detection of the protein of interest within the capillary. The capillary includes any convenient material, such as glass, plastic, silicon, fused silica, gel, or the like. In some embodiments, the method employs a plurality of capillaries. A plurality of capillaries enables multiple samples to be analyzed simultaneously.

The capillary can vary as to dimensions, width, depth and cross-section, as well as shape, being rounded, trapezoidal, rectangular, etc., for example. The capillary can be straight, rounded, serpentine, or the like. As described below, the length of the fluid path depends in part on factors such as sample size and the extent of sample separation required to resolve the protein of interest.

In some embodiments, the capillary includes a tube with a bore. In some embodiments, the method employs a plurality of capillaries. Suitable sizes include, but are not limited to, capillaries having internal diameters of about 10 to about 1000 μm, although more typically capillaries having internal diameters of about 25 to about 400 μm can be utilized. Smaller diameter capillaries use relatively low sample loads while the use of relatively large bore capillaries allows relatively high sample loads and can result in improved signal detection.

The capillaries can have varying lengths. Suitable lengths include, but are not limited to, capillaries of about 2 to 20 cm in length, although somewhat shorter and longer capillaries can be used. In some embodiments, the capillary is about 3, 4, 5, or 6 cms in length. Longer capillaries typically result in better separations and improved resolution of complex mixtures. Longer capillaries can be of particular use in resolving low abundance proteins of interest.

Generally, the capillaries are composed of fused silica, although plastic capillaries and PYREX (i.e., amorphous glass) can be utilized. As noted above, the capillaries do not need to have a round or tubular shape. Other shapes, so long as it is compatible with the methods described herein, may also be used.

In some embodiments, the capillary can be a channel. In some embodiments, the method employs a plurality of channels. In some embodiments, the capillary can be a channel in a microfluidic device. Microfluidics employs channels in a substrate to perform a wide variety of operations. The microfluidic devices can include one or a plurality of channels contoured into a surface of a substrate. The microfluidic device can be obtained from a solid inert substrate, and in some embodiments in the form of a chip. The dimensions of the microfluidic device are not critical, but in some embodiments the dimensions are on the order of about 100 μm to about 5 mm thick and approximately about 1 centimeter to about 20 centimeters on a side. Suitable sizes include, but are not limited to, channels having a depth of about 5 μm to about 200 μm, although more typically having a depth of about 20 μm to about 50 μm can be utilized. Smaller channels, such as micro or nanochannels can also be used, so long as they are compatible with the methods.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The following examples are provided to illustrate particular features of certain embodiments. However, the particular features described below should not be considered as limitations on the scope of the invention, but rather as examples from which equivalents will be recognized by those of ordinary skill in the art.

### EXAMPLES

### Example 1

### Development of Antibodies to HCPs for CE-Western

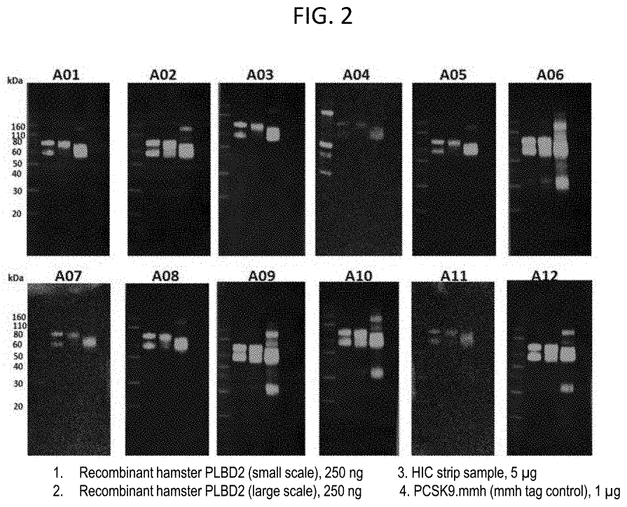

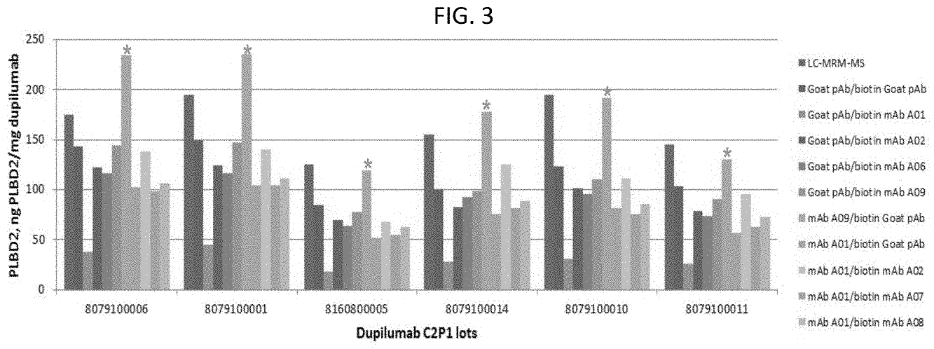

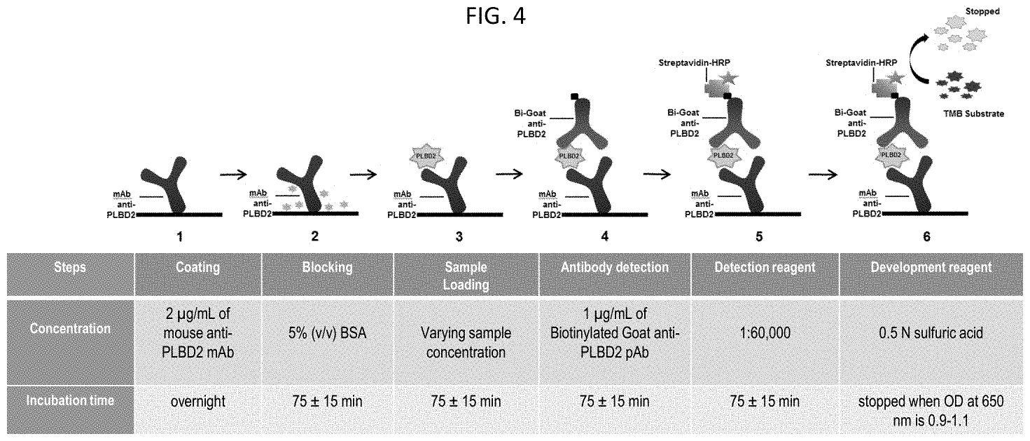

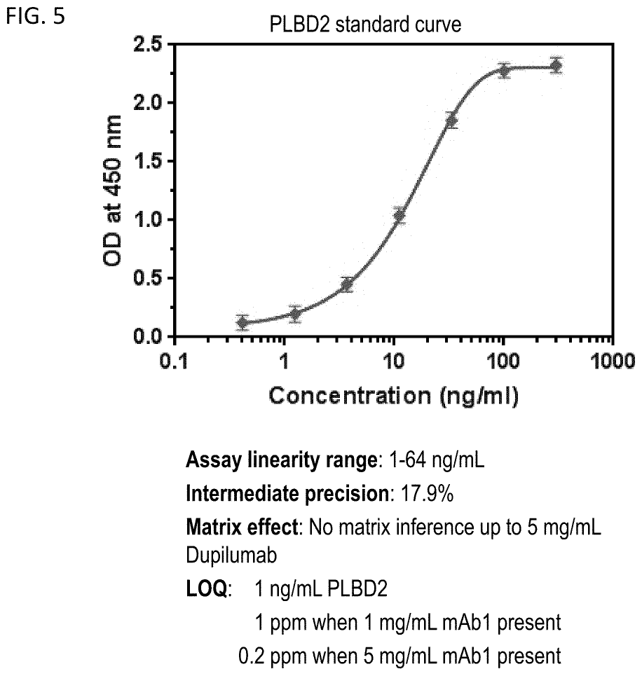

Goats and mice were immunized using recombinant PLBD2 or HIC strip to generate anti-PLBD2 pAbs and mAbs, respectively. Hybridomas were screened for specificity by western blot and 10 were selected for purification and biotinylation. Mature PLBD2 protein (−42 kDa) was not detected in any of the hybridomas. Antibodies targeting the N-terminus were identified. FIG. 2 is a set of digital images of Western blots using selected anti PLBD2 antibody preparations. FIG. 3 is a bar graph showing the measured PLBD2 level in antibody preparation samples with different combination of anti-PLBD2 antibodies. The ELISA measured amount is compared to the LC-MS data. From these studies, mAb09 coating and biotinylated goat pAb detection were selected for the final sandwich ELISA format. FIG. 4 is a schematic representation of a sandwich ELISA using selected anti-PLBD2 antibodies. FIG. 5 is a standard curve generated for a selected anti-PLBD2 antibody using the ELISA method.

### Example 2

### Separation and Detection with Size Based CE-Western

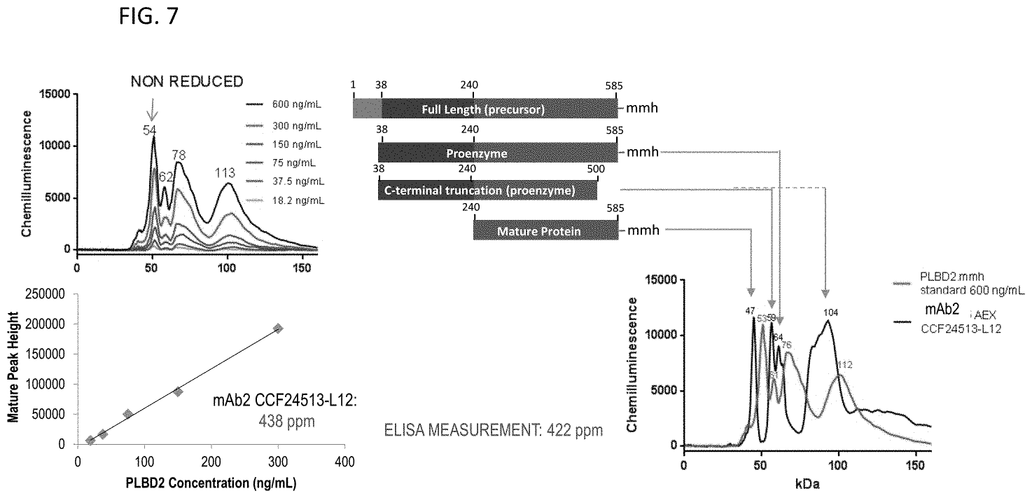

An antibody preparation that included the contaminant PLBD2 was analyzed by size-based CE-Western under reducing and non-reducing conditions (see FIG. 7). The graph shows a concentration dependent analysis of PLBD2, which demonstrates that the size based CE-western is comparable to an ELISA measurement for the detection and quantification of mAb preparation contaminants. In addition, unlike ELISA, because the contaminating proteins can be resolved by molecular weight, the individual species contributing to the overall contamination can be determined.

### Example 3

### Separation and Detection with Charge Based CE-Western

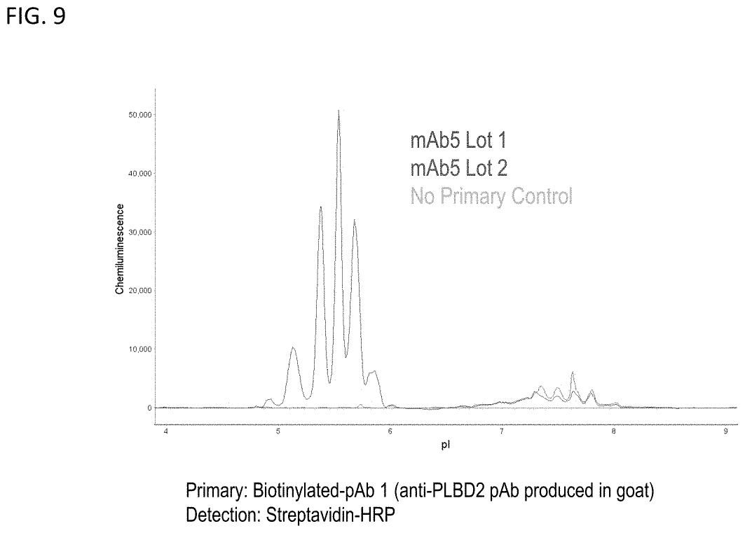

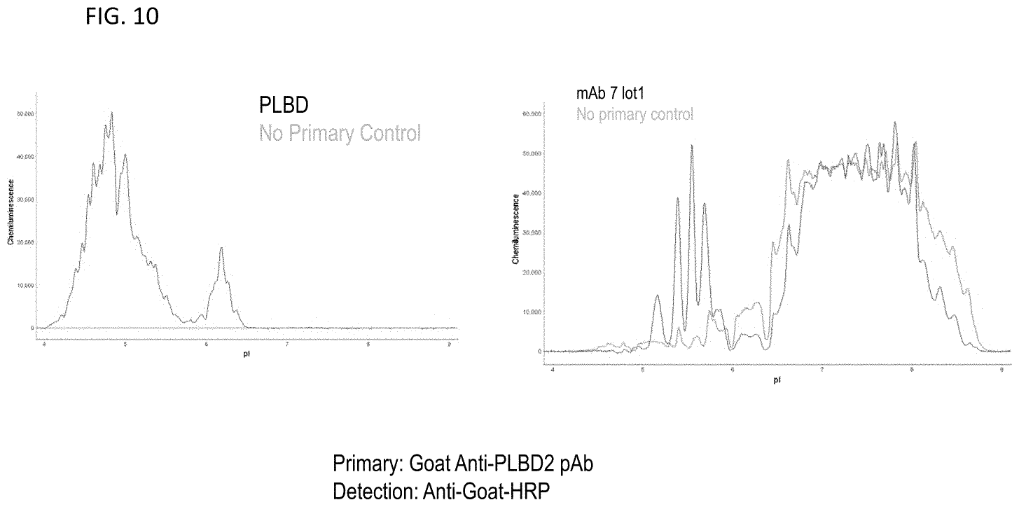

An antibody preparation that included the contaminant PLBD2 was analyzed by charge-based CE-Western (see FIGS. 9 and 10). FIG. 9 shows that the results of imaged cIEF-Western (icIEF) Charge Assay. PLBD2 is detected using the anti-PLBD2 pAb. PLBD2 is absent in the C2P2 process and that inclusion of the sample confirms that CE-western is specifically picking up the PLBD2 peaks in the 5-6 region. FIG. 10 shows that the results of imaged cIEF-Western (icIEF) Charge Assay. The native PLBD2 can be seen in the pH range of 5-6 in the figure on the right. This was detected from the mAb process demonstrating the ability of this method to selectively detect the PLBD2 from the process samples. In this charge mode, a specific monoclonal antibody to PLBD2 can be used to detect the process sample.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for detecting protein contaminants of interest in an antibody preparation sample, comprising:
separating protein components of a sample by a physical parameter in one or more capillaries using capillary electrophoresis;
immobilizing the protein components of the sample within the one or more capillaries;
contacting the protein components within the one or more capillaries with one or more primary antibodies that specifically bind to a protein contaminant of interest; and
detecting the binding of the one or more primary antibodies, thereby detecting protein contaminants of interest in an antibody preparation sample.

2. The method of claim 1, further comprising discriminating between variants of a protein contaminant of interest in an antibody preparation sample by the physical parameter.

3. The method of claim 1, wherein the one or more capillaries comprise a separation matrix.

4. The method of claim 3, wherein the separation matrix comprises carrier ampholytes.

5. The method of claim 4, wherein the physical parameter comprises charge.

6. The method of claim 3, wherein the separation matrix comprises a sieving matrix configured to separate proteins by molecular weight.

7. The method of claim 6, wherein the physical parameter comprises molecular weight.

8. The method of claim 1, wherein the one or more primary antibodies are labeled with a detectable label, and wherein detecting the binding of the one or more primary antibodies comprises detecting the detectable label.

9. The method of claim 1, wherein detecting the binding of the one or more primary antibodies comprises:
contacting the one or more primary antibodies with a secondary antibody that specifically binds at least one of the one or more primary antibodies, and wherein the secondary antibody has a detectable label; and
detecting the detectable label.

10. The method of claim 1, further comprising detecting and/or discriminating between charge or size variants of the protein contaminants of interest.

11. The method of claim 1, further comprising determining a relative or absolute amount of the protein contaminants of interest.

12. The method of claim 1, wherein the detectable label comprises a chemiluminescent label, a fluorescent label or a bioluminescent label.

13. The method of claim 1, wherein the sample includes an internal standard.

14. The method of claim 1, wherein the immobilizing comprises photo-immobilizing, chemically immobilizing, or thermally immobilizing.

15. The method of claim 1, wherein the one or more primary antibodies comprise polyclonal antibodies.

16. The method of claim 1, wherein the protein contaminants of interest comprise of PLBD2, CTSD, TIMP1, Acid Ceramidase (ASAH1), Lysosomal Acid Lipase (LAL),Annexin, Cathepsin B, Antileukoproteinase (ALP), or a fragment thereof.

17. A method for detecting and/or discriminating between protein contaminants of interest in an antibody preparation sample by a physical parameter, comprising: separating protein components of a sample by a physical parameter in one or more capillaries using capillary electrophoresis;
immobilizing the protein components of the sample within the one or more capillaries;
contacting the protein components within the one or more capillaries with a first primary antibody that specifically binds to a first protein contaminant of interest;
detecting the binding of the first primary antibody, thereby detecting the first antibody of interest;
contacting the protein components within the one or more capillaries with a second primary antibody that specifically binds to a second protein contaminant of interest; and
detecting the binding of the second primary antibody, thereby detecting the protein contaminants of interest and discriminating between the antibodies in a sample.

18. The method of claim 17, further comprising contacting the protein components within the one or more capillaries with a third primary antibody that specifically binds to a protein contaminant of interest; and
detecting the binding of the third primary antibody, thereby detecting the third protein contaminant of interest.

19. The method of claim 18, further comprising contacting the protein components within the one or more capillaries with one or more additional primary antibodies that specifically bind to one or more additional protein contaminants of interest;
detecting the binding of the one or more additional primary antibodies, thereby detecting the additional protein contaminants of interest.

20. The method of claim 17, further comprising discriminating between variants of a protein contaminant of interest in an antibody preparation sample by the physical parameter.

21. The method of claim 17, wherein the one or more capillaries comprise a separation matrix.

22. The method of claim 21, wherein the separation matrix comprises carrier ampholytes.

23. The method of claim 22, wherein the physical parameter comprises charge.

24. The method of claim 21, wherein the separation matrix comprises a sieving matrix configured to separate proteins by molecular weight.

25. The method of claim 24, wherein the physical parameter comprises molecular weight.

26. The method of claim 17, wherein the primary antibodies are labeled with a detectable label, and wherein detecting the binding of the primary antibodies comprises detecting the detectable label.

27. The method of claim 17, wherein detecting the binding of the primary antibodies comprises:
contacting the primary antibodies with a secondary antibody that specifically binds the primary antibodies, and wherein the secondary antibody has a detectable label; and
detecting the detectable label.

28. The method of claim 17, further comprising determining a relative or absolute amount of one or more of the protein contaminants of interest.

29. The method of claim 17, wherein the detectable label comprises a chemiluminescent label, a fluorescent label or a bioluminescent label.

30. The method of claim 17, wherein the sample includes an internal standard.

31. The method of claim 17, wherein the immobilizing comprises photo-immobilizing, chemically immobilizing, or thermally immobilizing.

32. The method of claim 17, wherein the protein contaminants of interest comprise of PLBD2, CTSD, TIMP1, Acid Ceramidase (ASAH1), Lysosomal Acid Lipase (LAL),Annexin, Cathepsin B, Antileukoproteinase (ALP), or a fragment thereof.
